(19) United States
(12) Patent Application Publication
(10) Pub. No.: US 2004/0132174 A1
(43) Pub. Date: Jul. 8, 2004

(54) Perfusion incubator

(76) Inventors: Allan Joseph Hilling Smith, Queensland (AU); David Sean O'Brien, Queensland (AU); David Paul Michel, Queensland (AU); Paul Chan, Queensland (AU)

Correspondence Address: BRINKS HOFER GILSON & LIONE, P.O. BOX 10395, CHICAGO, IL, 60610, US

(21) Appl. No.: 10/662,981

(22) Filed: Sep. 15, 2003

Related U.S. Application Data
(63) Continuation-in-part of application No. 09/819,407, filed on Mar. 28, 2001, now Pat. No. 6,627,435.
(60) Provisional application No. 60/412,423, filed on Sep. 21, 2002.

(30) Foreign Application Priority Data
Mar. 28, 2000 (AU) PQ 6530
Sep. 17, 2002 (AU) 2002951423

(51) Int. Cl. C12M 3/00

(52) U.S. Cl. 435/293.1; 435/292.1; 359/398

(57) ABSTRACT

A perfusion incubator having an incubation well assembly is provided. A peristaltic pump may supply medium from a medium supply and pass it through a medium conditioning unit before supplying the medium to the incubation well assembly. Each well assembly may have an upper portion and a lower portion, a medium inlet and a medium outlet. The medium inlet may be positioned at a mid point in the well and the medium outlet is positioned above the medium inlet. An embryo may reside in the lower portion of the well. The well assembly may be formed from a permeable material through which a gas, such as carbon dioxide and/or oxygen, can diffuse.

This application is a continuation-in-part of U.S. Nonprovisional application Ser. No. 09/819,407, filed Mar. 28, 2001, entitled “Perfusion Incubator,” the contents of which are incorporated by reference in their entirety, which claimed priority to Australian Patent Application No. PQ 6530, filed Mar. 28, 2000, the contents of which are incorporated by reference in their entirety, and claims priority to U.S. Provisional Application Serial No. 60/412,423 entitled “Improved Perfusion Incubator” filed on Sep. 17, 2002, which is incorporated by reference in its entirety, and Australian Provisional Application Serial No. 2002951423 entitled “Improved Perfusion Incubator” filed on September 17, 2002, which is incorporated by reference in its entirety.

### TECHNICAL FIELD

This invention generally relates to perfusion incubators for living cells, particularly the living embryos of mammalian species.

### BACKGROUND

Some cells growing in a liquid medium produce exogenous and growth factors that surround the cell in the liquid medium. In the growing of such cells in vitro, it is preferable not to immediately flush these exogenous and growth factors away from the living cell when replenishing the medium around the cell.

It is an objective of the present invention to provide an incubator well and an incubator device that can provide favorable conditions for the growing of cells, including embryos.

### BRIEF DESCRIPTION

The perfusion incubator may include a medium supply, a medium conditioning unit, at least one well assembly having an upper portion and a lower portion, a well assembly heating unit, a peristaltic pump, and a medium collection unit. Each well may have a medium inlet and a medium outlet. The medium inlet may be positioned at a mid point in the well, and each medium outlet may be positioned at a point in the well above its respective medium inlet. The embryo to be cultured may be placed in a lower portion of the well. The medium inlet may be connected to the medium supply via the peristaltic pump, and the medium outlet may be connected to the medium collection unit. Thus, the embryo may be cultured in the lower portion of the well when a liquid medium is passed through the well.

An illumination device may be included so the embryo being cultured in the lower portion of the well can be observed by a microscope and the like. A microscope mount may be associated with the perfusion incubator for this purpose.

Each well assembly may include means to provide a flow path from the medium inlet to the medium outlet within the well so that medium flow is not directed directly at the lower portion of the well, instead being tangential to the lower portion. Each medium inlet may be positioned to allow a tangential entry of medium to the well at a mid point in the well, and each medium outlet may be positioned above the medium inlet to allow the exit of medium from the well. This construction may provide a slow vortex flow of medium in the lower portion of the well, thus replenishing the medium surrounding the embryo without directly passing medium over the embryo.

Each well can have a stepped side-wall defining an upper chamber and a smaller diameter lower chamber. Each well may also have a lid, which extends partially into the upper chamber with an interference fit. Each lid may be made of a substantially transparent material, so as to allow for viewing of the embryo in the lower chamber by means of a microscope and the like. The well assembly may be wholly or partially transparent so that the embryo can be illuminated from below.

The peristaltic pump may provide a flow rate of medium through each well assembly of from about 1 microlitre per hour to 10,000 microlitres per hour.

The medium conditioning unit may include means to regulate the temperature of the medium and means to regulate the pH and growing condition of the medium, pH regulation may be provided through the perfusion of one or more gasses into the liquid medium.

The means to regulate the temperature of the medium may be operated at a temperature above the operating temperature of the well. The temperature difference between the medium entering the well and the medium contained in the well can increase the solubility of gases in the liquid medium contained in the well, thus reducing the likelihood that gas will be liberated while the medium is contained in the well. In one aspect, the temperature difference can substantially prevent the formation of gas bubbles in the liquid medium present in the well.

The means to regulate the pH and growing condition of the medium may include a means to condition the medium with a gas. Conditioning may include the diffusion or perfusion of a gas, such as carbon dioxide and/or oxygen, into the liquid medium.

Thus, at least a portion of the well assembly may be formed from a permeable material and at least a portion of the well may be surrounded by a lumen into which a gas may be supplied. Any permeable material compatible with the growth of the embryo, such as a silicone elastomer, may be used that provides for gases, such as carbon dioxide and/or oxygen, to diffuse into the well, thus providing pH and growing condition control.

All or a portion of the medium inlet tube may also be formed from the same or another permeable material as the well assembly. The portion of the medium inlet tube formed from the permeable material may be wholly or partially surrounded by a concentric jacket into which a gas, such as carbon dioxide, may be introduced. Thus, the medium entering the well through the medium inlet tube may be conditioned with the gas.

The gas may be provided in a counter current flow and supplied into a lumen below the well in the well assembly from an aperture in a heater plate that may support the well assembly. The heater plate may maintain the temperature of the well and serve as the well assembly heating unit discussed above.

Because the permeable material may be selected to be substantially translucent, the body of the well may be illuminated from below the embryo being cultured, thus enabling the embryo to be viewed from above by a microscope and the like.

In another aspect, the perfusion incubator may include a perfusion incubator well assembly having a body, the body being formed from a material through which a gas, such as carbon dioxide and/or oxygen, can diffuse; at least one well in the body, the at least one well having a stepped side-wall defining an upper chamber and a smaller diameter lower chamber, and a lid. The perfusion incubator may also include a medium inlet to the at least one well and a medium outlet from the at least one well. The medium inlet may be positioned so as to allow the tangential entry of medium to the well at a lower portion of the upper chamber. The medium outlet may be positioned above the medium inlet. A lumen in the body may be adapted to provide a diffusion path for gas into the at least one well.

A lid may extend partially into the upper chamber and be made of a substantially transparent material so as to allow the viewing of an embryo residing in the lower chamber. The body may also be made from a substantially translucent material so that illumination provided below the at least one well may be viewed through the lid.

The medium inlet to and the medium outlet from the at least one well may be formed by ducts or apertures formed in the body.

The body of the well assembly may be formed from a silicone elastomeric material.

Preferably the lumen is open to a base of the well assembly. The gas supplied to the lumen may be a mixture including oxygen, carbon dioxide, or nitrogen.

In a further form, the invention may include a perfusion incubator well assembly having a body and a lid, the body including at least one well therein and being formed from a material through which a gas, such as carbon dioxide and/or oxygen, can diffuse.

In a further form, the invention may include a perfusion incubator and well assembly with at least one well, a peristaltic pump to supply medium from a medium supply through a medium conditioning unit to the well assembly, and a medium outlet from the well assembly, whereby on the placing of an embryo to be cultured in the at least one well and flowing medium through the well, culturing of the embryo can occur.

The perfusion incubator may be a system based on the perfusion of a liquid culture medium with a gas and may provide a suitable environment for the production, development, and storage of pre-implantation embryos from mammalian species. The system may include culture wells that may be maintained at a pre-set temperature while a gas enriched medium is perfused over the embryos.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like references numerals designate corresponding parts throughout the different views.

FIG. 1 shows a schematic view of the tubes and well portions of a perfusion incubator system according to one embodiment the present invention:

FIG. 2 shows an isometric view of a perfusion incubator according to one embodiment of the invention but without the tubes and well;

FIG. 3 shows a detail plan view of part of the perfusion incubator according to FIG. 2;

FIG. 4 shows a detail of part of the peristaltic pump portion of the perfusion incubator according to FIG. 2;

FIG. 5 shows a detail of part of the well section of the perfusion incubator according to FIG. 2;

FIG. 6 shows a cross sectional view of a culture chamber or well assembly for the perfusion incubator of the embodiment shown in FIG. 2; and

FIG. 7 shows an underneath perspective view of a well assembly for the perfusion incubator of the embodiment shown in FIG. 2.

### DETAILED DESCRIPTION

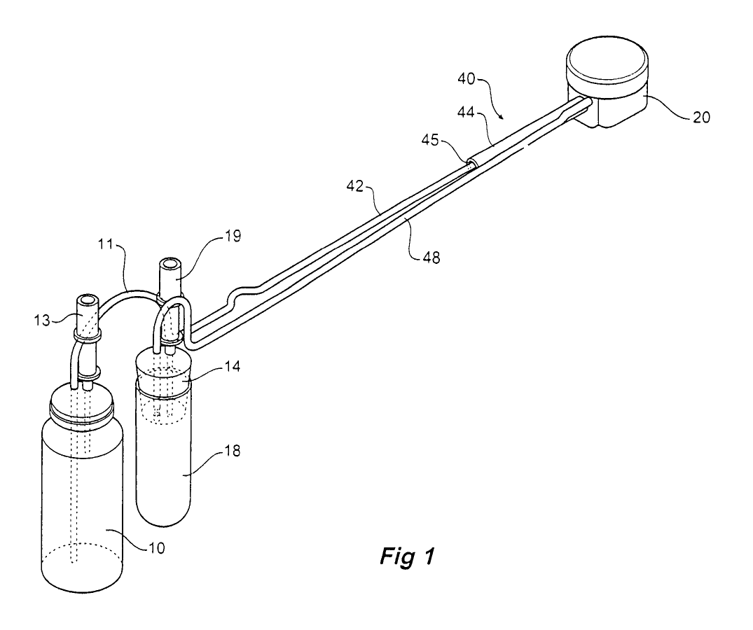

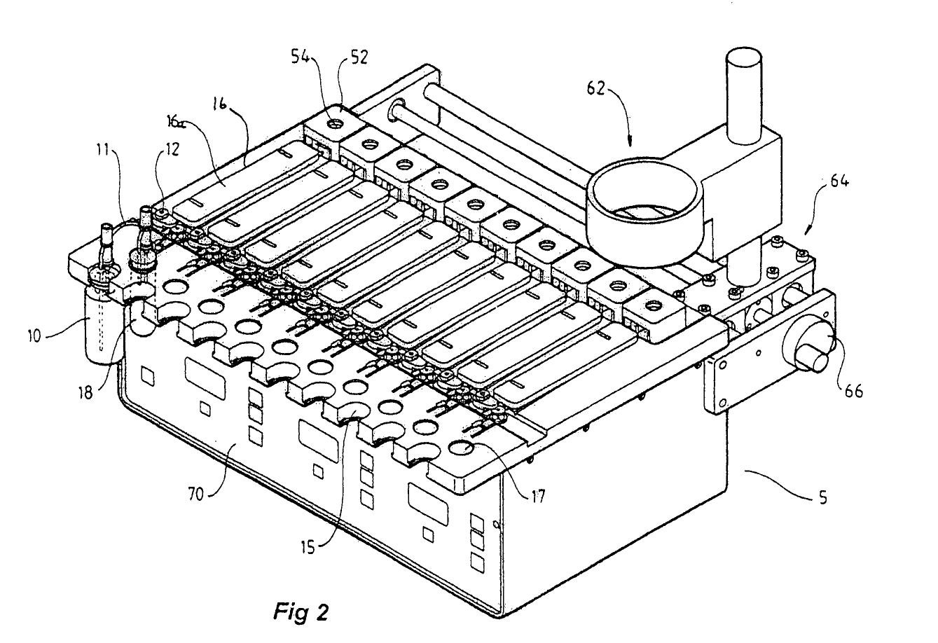

FIG. 1 shows the replaceable and/or disposable part of a perfusion incubator according to this invention. FIG. 2 shows the device into which the replaceable or disposable portion shown in FIG. 1 may be fitted.

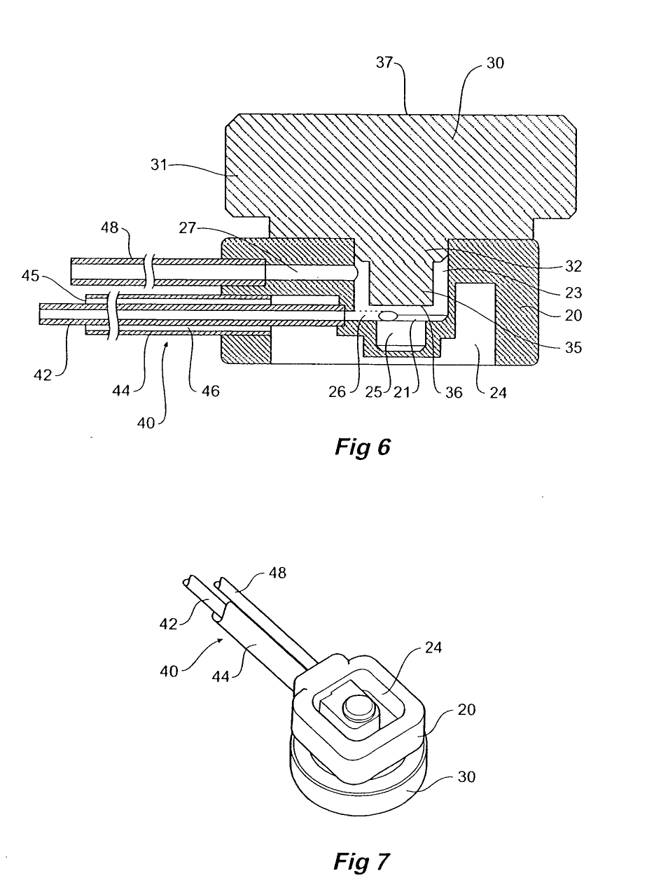

The perfusion incubator includes a well assembly 20 which will be discussed in greater detail in relation to FIGS. 6 and 7. Extending to the well assembly 20 is a medium inlet tube 42 and extending from the well assembly 20 is a medium outlet tube 48. The medium inlet tube 42 may include a coaxial portion 40 in which there is an outer tube 44 surrounding the medium inlet tube 42. Gas may enter the lumen 24 (shown in FIGS. 6 and 7) under the well assembly 20 and pass from lumen 24, under the well assembly 20, to the annular space 46 (shown in FIG. 6) between the medium tube 42 and the outer tube 44, and exit from the end 45 of the annular space 46.

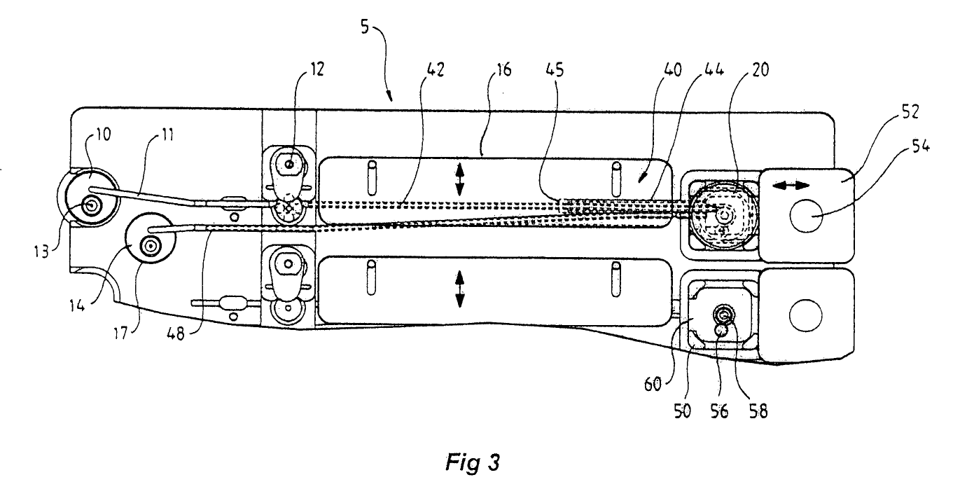

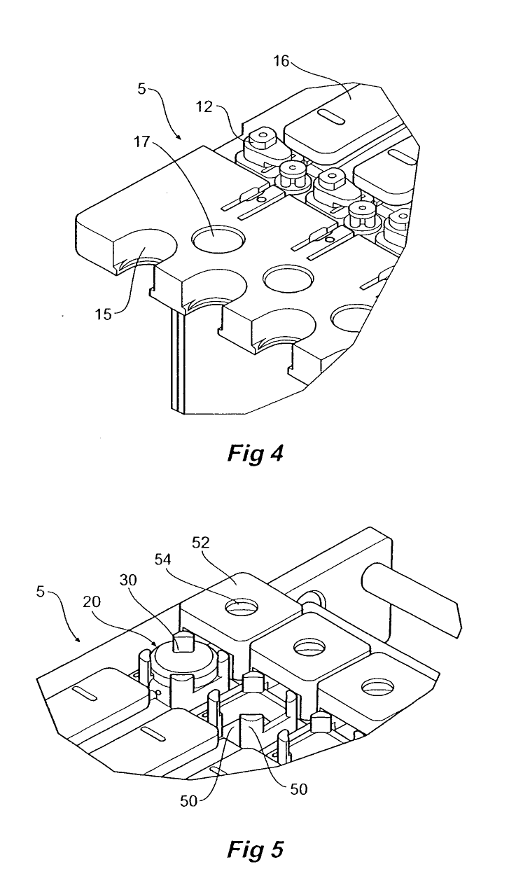

A liquid medium may be supplied from a medium bottle 10 and may travel by means of a pipe 11 to a peristaltic pump 12 (shown in FIGS. 2 and 3). The medium may be pumped by the peristaltic pump through a medium conditioning unit 16 (shown in FIG. 2) to the well assembly 20. A very fine pore filter 13 on the medium supply 10 may allow air to enter the supply as medium is released. The pore diameter of the filter may be selected to prevent bacteria or other contaminants from entering the medium supply 10. The medium supply may be any suitable container, such as a bottle, and the like.

Waste medium exiting the well assembly 20 through a waste medium line 48 may enter a medium collection unit 18, which may be fitted onto the bung 14. Again, a very fine pore filter 19 on the medium waste tube or collection unit 18 may allow air to be displaced from the unit as medium is drained into it, while preventing bacteria or other contaminants from entering. The medium collection unit may be any suitable container, such as a bottle, and the like. The medium supply may be supported by bracket 15 on the perfusion incubator device. The medium collection unit 18 may be supported in the aperture 17 in the perfusion incubator device.

In the region between the peristaltic pump 12 and the chamber 20, the inlet tube 42 may pass through the medium conditioning unit 16 which may include a heater unit covered by lid 16a. The lid 16a can slide sideways to enable placement of the inlet tube through the medium conditioning unit.

Each well assembly 20 can be received in a carrier 50 (FIGS. 3 and 5) and may be covered by an individual opaque covering lid 52. The covering lid may have an aperture 54 to enable viewing of the well assembly under the lid. In FIGS. 3 and 5, the lids are shown slid back to reveal the well assembly 20 in the carrier 50.

As can be seen in FIG. 3, in the carrier 50 there may be a gas outlet 56 and a light emitting diode 58. The gas outlet may provide gas from a gas supply (not shown) into the lumen 24 (shown in FIGS. 6 and 7) of the well assembly 20, as will be discussed further below. The base 60 of the well carrier 50 may be heated to provide a substantially constant temperature for the well assembly 20.

As can be seen in FIG. 2, a microscope holder 62 may be adapted to travel by means of a carrier 64 along the line of wells so that the contents of each well can be viewed through the respective aperture 54 in the respective covering lid. Handle 66 may be provided to move carrier 64 and hence the microscope between the wells. Control panel 70 on the front of the device may provide the necessary control facilities for setting and monitoring the incubation conditions.

As can be seen in the cross sectional side view in FIG. 6 and the underneath perspective view shown in FIG. 7, the well assembly 20 may have a well generally shown as 21 and a lid 30. The well 21 may have an internal side wall with a stepped cross section to define a larger upper section 23 into which the lid fits and a smaller diameter lower section 25. The medium inlet 26 may extend from medium inlet tube 42 and enter the well at the bottom of the upper section 23 in a tangential manner. The medium outlet 27, which may extend to the medium outlet tube 48, may exit near the top of the upper section 23. One or more cells to be cultured may be placed in the lower section 25.

The lid 30 may have an upper portion 31 to enable gripping for placement and removal of the lid and may be of a size which can be gripped by the user. A central portion 32 of the lid 30 may be adapted to fit into the upper section 23 of the well with an interference fit to provide sealing of the lid into the chamber. The lower portion 35 of the lid 30 may be adapted to extend down into the upper section 23 of the well, but also may allow for medium to flow around the lower portion of the lid and towards the medium outlet 27. In this fashion, the lower portion 35 may assist in maintaining the slow vortex effect due to the tangential entry of the medium at the inlet 26. The lower surface 36 and the upper surface 37 of the lid 30 may be finely polished so that viewing through the lid enables the one or more cells being cultured in the lower section 25 of the well assembly 20 to be observed.

Lumen 24 may be formed into the well assembly 20 and may surround at least a portion of the well 21. The lumen 24 may be open to the bottom of the well assembly 20. Gas supplied into the lumen 24 can diffuse into the well through a permeable material from which the chamber 20 may be constructed. The permeable material may be a silicone elastomer.

Gas may also flow from the lumen 24 into the coaxial tube arrangement generally shown as 40. The coaxial tube arrangement 40 may have an inner tube 42 made from a permeable material and an outer coaxial tube 44. The gas that flows out of the lumen 24 may flow through the annular space 46 between the inner tube 42 and the outer tube 44. The inner tube 42 may also be made of a permeable material, such as a silicone elastomer, which allows for the diffusion of a gas from the annular space 46 through the tube wall and into the liquid medium flowing through the medium inlet tube. Hence, the medium may be conditioned during its travel to the well assembly. In one aspect, conditioning includes pH regulation. The inner tube 42 may be a continuation of the medium inlet 26 and may supply the liquid medium to the well. The perfusion incubator and/or the well assembly may include fewer or additional components.

The gas supplied to the lumen 24 may be a mixture including carbon dioxide, nitrogen, and/or oxygen. The combination of gases may be varied depending upon the type of cell being cultured and may also be varied during the culturing process depending on the growth phase of the cell. The oxygen may sustain cell growth while the carbon dioxide may regulate the pH of the liquid medium surrounding the cell. For instance, the carbon dioxide concentration may range from 0 to about 20% by volume and the oxygen concentration may range from 0 to about 20% by volume, with the balance being nitrogen.

Waste medium from the well may exit through medium outlet 27 and flow into the medium outlet tube 48.

The lid 30 of the culture well may be made of a plastic material, such as a polycarbonate, and may have highly polished upper and lower surfaces. A high output light emitting diode 58 (FIG. 3) may be placed under each culture well. A set of ten culture wells may be contained on a heating block.

A significant factor in the ability to grow a healthy embryo can be the ability to directly observe the growth pattern of the embryo and make changes in the medium and other growth parameters during the development of the embryo. Each of the well assemblies 20 may have a lid 52 including a viewing aperture 54. Above the heater block 60, upon which the well assemblies 20 may be placed, may reside a microscope holder 62. When a microscope is in place and the light emitting diode (LED) 58 beneath the well assembly 20 is turned on, an operator can observe the cell or embryos in the well directly in situ. A motor driven system or a manual system 66 to position the microscope over each well may be included.

The motor driven system may be provided to position the microscope over each consecutive well assembly, such as at one minute intervals, which allows for digital time lapse photography to capture the growth pattern of the individual embryos residing in each well. The illumination beneath each well may be turned on only as required to capture an image. Light output from the LED may be in the orange-yellow band, thus being of low energy, but providing high contrast. In addition, it is preferable that the light emitting source contains no ultra-violet radiation, as this may damage the embryos.

Embryos for culture may be placed in the lower portion of the well assemblies where they may reside in the margin between the horizontal and vertical axis of the lower section. During culturing, the embryo is not substantially dislodged from the lower portion of the well assembly by the flow of the liquid medium.

A liquid culture medium may be introduced above the lower section as a tangential flow to the inner surface of the well and removed from the top of the well. This can create an upward, slow moving vortex, which may displace medium in the lower section of the well. Without this vortex action, medium exchange in the bottom section of the well is limited to diffusion, which can result in embryo death or at best, embryos with very poor morphology that are unsuitable for implantation.

A preferable feature of the wells is their ability to provide a vortex action that exchanges the medium in the bottom section of the well. This action can be observed by injecting small amounts of dye into the medium and following the fluid path. Thus, by not placing the embryos directly in the fluid path, the exogenous and growth factors excreted by the embryos are not immediately flushed away. By placing the embryo in a lower portion of the well, where the medium exchange rate is less than the true flow rate, the exogenous and growth factors substantially remain in the proximity of the embryo for at least a short time.

In operation mode, with the lids in place, there may be an outgassing of the solubilized gasses from the liquid medium. This outgassing can lead to the formation of small bubbles in the liquid medium. If small bubbles form in the liquid medium residing in the well, the bubbles may surround and isolate the developing embryo from the medium, resulting in the death of the embryo. Once formed, these minute bubbles may not be able to be substantially dislodged from the embryo at the flow rates used in the device. The flow rate used per well can vary from about 1 microlitre per hour up to 10,000 microlitres per hour when in flush mode.

To overcome this bubble problem, liquid medium above the operating temperature of the well may be equilibrated with a gas mixture. This equilibration prior to the introduction of the liquid medium into the well allows for an increase in the gas solubility of the liquid medium because of the temperature drop that occurs when the medium enters the well. This increased solubility of the gas in the cooling liquid medium may substantially prevent gas bubbles from forming in the liquid medium within the well. Preferably, the liquid medium is introduced to the well at a temperature that is from 0.05° to 1.5° C. above the temperature of the medium in the well and more preferably from 0.2° to 0.8° C. above the temperature of the medium in the well. At present, it is especially preferred that the liquid medium is introduced to the well at a temperature that is from 0.4° to 0.6° C. above the temperature of the medium in the well. In another aspect, the liquid medium introduced to the well is about 0.5° C. above that of the medium in the well.

The outgassing may also be reduced by placing the peristaltic pump before the well, as opposed to after the well. In this fashion, the liquid medium is introduced to the well under positive pressure as opposed to being removed from the well under a vacuum.

Once the embryos are ready for implantation, they can be perfused with cryoprotectant and the embryo can then be extracted from the lower portion of the well and frozen in liquid nitrogen. Other methods may be used to harvest the embryos from the wells.

The perfusion incubator may be used to culture mammalian cells by perfusing a liquid medium over the cells by a slow vortex action. The liquid medium may be pre-heated and gassed in transit to the well by the perfusion of a gas into the medium through tubing made from a permeable material. Counter-current gas flow may be used so that the medium flowing into the well encounters a gas having elevated concentrations of carbon dioxide and/or oxygen. The process of gassing the medium by gas perfusion through the tubing may eliminate the need to humidify the gas. The pH of the medium may be maintained in the porous well, such as a well having a base made from a silicone elastomer, by the perfusion of one or more gases through the base of the well and into the liquid medium.

The incubator may have ten individual peristaltic pumps pulling fluid from ten individual un-gassed and un-heated medium supplies. The medium flowing through a length of a permeable tubing exposed to a gas along the medium conditioning section, which may be heated, can allow the equilibration of both gas concentration and temperature prior to the medium entering the well.

pH testing, using a one hundred millimetre length of coaxial system with a 2 mm outer diameter and a 1 mm inner diameter silicone tube carrying the medium at 500 μl per hour may give pH values of about 7.3 to about 7.4. Medium flow rates can be from about 1 to 10,000 μl per hour, but flow rates above about 500 μl per hour are preferably used to flush the system. The tube or tubes connecting the medium supply and the well may be retained in a groove in the heated top plate of the medium conditioning unit 16 by a sliding clear polycarbonate door.

The lid of the well may be a polycarbonate and may seal with the well by an interference fit. Other sealing methods may be used. The well may have a recess or lumen within the base through which a gas will flow up from within the surrounding heating chamber before exiting via the coaxial medium inlet tubing set. Incoming medium may be gassed by the counter-current gas flow in the outer tube of the coaxial medium inlet tube. Waste medium may exit alongside the inlet tubing and empty into a collection unit, such as a 12 mL test tube at the front of the machine. Gas may be vented to the air after passing through the coaxial medium inlet tubing.

The medium inlet tube may be connected to the medium by a length of rigid plastic, such as a polytetrafluoroethylene (PTFE) containing plastic, which passes through a silicone stopper on the medium supply. The medium bottle may be maintained at atmospheric pressure by inserting a needle with a Luer lock 0.22μ sterile filter through the silicone stopper. Similarly, the medium outlet may be connected to the collection unit by a length of rigid plastic passed through a silicone stopper inserted in the medium collection unit. The medium collection unit may be maintained at atmospheric pressure by the insertion of a needle with a Luer lock 0.22μ sterile filter through the silicone stopper.

Each well may be covered by an individual plastic lid, which slides fore and aft. This lid may push down on the well lid to ensure that the well is correctly seated and that the well makes good contact with the heated base.

Control of the device may be provided by an internal microprocessor and/or an external PC and the like.

The device may have a control on a front panel to set a temperature for the inlet tubes, such as from about 35 to about 40° C., another control to set the temperature for the culture well chambers, such as from about 35 to about 40° C., and a further control to set gas flow, such as from about 30 to about 50 mL/min. Each control may have an associated display to indicate the current value and the set value. The total gas flow may be divided equally between all tracks. For ten tracks, a gas flow of about 30 mL/min could give a flow of about 3 mL/min/track. In one aspect, the about 3 mL/min/track has been found to be sufficient to maintain the desired pH.

Protocols to control the speed and duty cycle times of the ten individual peristaltic pumps may be entered onto a graphical user interface on a PC and the like connected to the device. The external PC also may be used to log temperatures, flow-rates, and duty-cycle times for individual channels.

A microscope carrier may run along the rear support rail and may be hand driven from the right or the left. A LED may be situated below each culture chamber to illuminate the well contents for microscopic viewing. Each LED may be activated by two controls on the left side of the device. In one aspect, a first control may be provided that has ten select points and allows the user to select a single LED, while a second control may allow intensity setting of the selected LED to be varied. The second control may have 11 select points with 1 being off and 11 being the maximum intensity.

As one of ordinary skill in the art will recognize from the provided description, figures, and examples, modifications and changes can be made to the preferred embodiments of the invention without departing from the scope of the invention defined by the following claims and their equivalents. The examples are given for illustration only and not for limitation.

What is claimed is:

1. A perfusion incubator, for culturing living cells, comprising:
a medium supply;
at least one well assembly with a well having an upper portion and a lower portion; and
a peristaltic pump,
where each well assembly includes a medium inlet and a medium outlet, each medium outlet is positioned above the medium inlet, and the medium inlet is connected to the medium supply via the peristaltic pump.

2. A perfusion incubator as in claim 1, further comprising an illumination device so that the lower portion of the well assembly can be observed by means of a microscope.

3. A perfusion incubator as in claim 2, further comprising a microscope mount associated with the perfusion incubator.

4. A perfusion incubator as in claim 1, where each well includes a means to provide a flow path from the medium inlet to the medium outlet within the well so that medium flow is tangential to the lower portion of the well.

5. A perfusion incubator as in claim 1, where each medium inlet is positioned so as to allow a tangential entry of medium to the well at a mid point in the well, where the flow of medium in the well is formed by this construction into a vortex.

6. A perfusion incubator as in claim 1, where each well has a stepped side wall defining an upper chamber and a smaller diameter lower chamber.

7. A perfusion incubator as in claim 6, where each well has a lid that extends partially into the upper chamber.

8. A perfusion incubator as in claim 7, where each lid is made of a substantially transparent material.

9. A perfusion incubator as in claim 1, where at least a portion of the well assembly is made from a substantially transparent material.

10. A perfusion incubator as in claim 1, where the peristaltic pump provides a flow rate of medium through each well of from 1 microlitre per hour to 10,000 microlitres per hour.

11. A perfusion incubator as in claim 1, further comprising a medium conditioning unit, where the medium conditioning unit includes means to regulate the temperature of a medium and means to regulate the pH of the medium.

12. A perfusion incubator as in claim 11, where the means to regulate the temperature of the medium is operated at a temperature of from 0.05° to 1.5° C. above the operating temperature of a well assembly.

13. A perfusion incubator as in claim 11, where the means to regulate the pH of the medium includes means to diffuse a gas into the medium.

14. A perfusion incubator as in claim 11, where at least part of the well assembly is formed from a silicone elastomeric material and at least part of the well is surrounded by a lumen in the well assembly into which a gas is supplied.

15. A perfusion incubator as in claim 1, where at least a portion of the medium inlet tube is formed from a silicone elastomeric material and at least a portion of the medium inlet tube formed from the silicone elastomeric material is surrounded by a jacket into which a gas is provided, where at least a portion of the gas diffuses through the medium inlet tube and into the medium.

16. A perfusion incubator well assembly comprising:
a body, the body being formed from a material through which a gas can diffuse;
at least one well in the body, the at least one well having a stepped side wall defining an upper chamber and a smaller diameter lower chamber;
a medium inlet to the at least one well;
a medium outlet from the at least one well, where the medium inlet is positioned to allow tangential entry of medium to the at least one well at a lower portion of the upper chamber, and the medium outlet is positioned above the medium inlet; and
a lumen in the body, where the lumen provides a path for a gas to diffuse into the at least one well.

17. A perfusion incubator well assembly as in claim 16, where the lumen is open to a base of the well assembly.

18. A perfusion incubator well assembly as in claim 16, where a silicone elastomeric material forms at least a portion of the body of the well assembly.

19. A perfusion incubator well assembly as in claim 16, further comprising a lid that extends partially into the upper chamber, where the lid is made of a substantially transparent material.

20. A perfusion incubator well assembly comprising:
a body that includes at least one well, where a material through which a gas can diffuse forms at least a portion of the body;
a medium inlet to the at least one well;
a medium outlet from the at least one well, where the medium inlet is positioned to allow tangential entry of medium to the at least one well at a lower portion of the upper chamber, and the medium outlet is positioned above the medium inlet.

21. A perfusion incubator well assembly as in claim 20, where a silicone elastomeric material forms the body of the well assembly.

22. A perfusion incubator well assembly as in claim 20, where the lid is a substantially transparent material.

23. A perfusion incubator well assembly as in claim 20, where the at least one well includes a stepped side wall defining an upper chamber and a smaller diameter lower chamber;
a medium inlet to the at least one well and a medium outlet from the at least one well, where the medium inlet is positioned to allow tangential entry of the medium to the at least one well at a lower portion of the upper chamber, and the medium outlet is positioned above the medium inlet; and
a lumen in the body, the lumen adapted to provide a path for a gas to diffuse into the at least one well.

24. A perfusion incubator comprising:
at least one well assembly;
a peristaltic pump; and
a medium inlet tube, the inlet tube providing fluid communication between the peristaltic pump and the at least one well assembly, where at least a portion of the medium inlet tube is formed from a material through which a gas can diffuse,
and at least a portion of the medium inlet tube formed from the material through which a gas can diffuse is surrounded by a jacket into which a gas is provided, where at least a portion of the gas diffuses through the medium inlet tube and into the medium.

25. A perfusion incubator as in claim 24, where the at least one well further comprises:
a medium inlet in fluid communication with the medium inlet tube, where the medium inlet is positioned at a mid point in a well of the at least one well assembly; and
a medium outlet, the medium outlet positioned above the medium inlet.

26. A perfusion incubator as in claim 24, where the at least one well assembly has at least one well having a stepped side wall defining an upper chamber and a smaller diameter lower chamber.

27. A perfusion incubator as in claim 26, where an embryo resides in the smaller diameter lower camber.
